(19) United States
(12) Patent Application Publication
(10) Pub. No.: US 2006/0183112 A1
(43) Pub. Date: Aug. 17, 2006

(54) Method of separating biomolecules using nanopore

(76) Inventors: Jun-hong Min, Yongin-si (KR); Su-hyeon Kim, Seoul (KR); In-ho Lee, Yongin-si (KR); Kui-hyun Kim, Daejeon-si (KR); Seung-yeon Yang, Seongnam-si (KR)

Correspondence Address: CANTOR COLBURN, LLP, 55 GRIFFIN ROAD SOUTH, BLOOMFIELD, CT, 06002, US

(21) Appl. No.: 11/335,246

(22) Filed: Jan. 19, 2006

(30) Foreign Application Priority Data
Jan. 20, 2005 (KR) 10-2005-0005530

(51) Int. Cl. C12Q 1/70; C12Q 1/68

(52) U.S. Cl. 435/5; 435/6; 204/450

(57) ABSTRACT

Provided is a method of separating particles, the method comprising: forming a first chamber and a second chamber separated by an interface with a pore, wherein the first and second chambers have electrodes with different polarities; placing particles to which a target biomolecule is bound from particles to which the target biomolecule is not bound in the first chamber; applying a voltage which has the same polarity as that of the target biomolecule to the electrode of the first chamber, and a voltage which has an opposite charge to that of the target biomolecule to the electrode of the second chamber; and translocating only the particles to which the target biomolecule is bound from the first chamber to the second chamber through the pore. Conventionally, the size of a pore is used to separate biomolecules. However, effective separation is difficult to achieve because the manufacture of a pore with a diameter of less than 10 nm, small enough to separate biomolecule, is not easy. Therefore, signal separation and data analysis must be required. However, in the present method, physical movement induced by the charge of biomolecules is used to effectively separate the biomolecules, thus obtaining a high signal to noise ratio. As a result, additional data analysis is not required.

### CROSS-REFERENCE TO RELATED PATENT APPLICATIONS

This application claims the benefit of Korean Patent Application No. 10-2005-0005530, filed on Jan. 20, 2005, in the Korean Intellectual Property Office, the disclosure of which is incorporated herein in its entirety by reference.

### BACKGROUND OF THE INVENTION

1. Field of the Invention

The present invention relates to method of separating and detecting biomolecules, and more particularly, to method of separating and detecting particles bound with biomolecules using a nanopore.

2. Description of the Related Art

Many methods of detecting binding and hybridization of a target sample in a sample have been developed. Of these, a method using a nanopore is a bio-pore mimicking system and an ultra sensitive DNA detection system, and the use of the method can realize DNA sequencing in theory.

U.S. Pat. No. 6,362,002 entitled “Characterization of Individual Polymer Molecules Based on Monomer-interface Interactions” in the name of the University of Harvard discloses a method of detecting a double-stranded nucleic acid by providing an interface between two pools of a medium, the interface having a channel that allows passage of a single-stranded nucleic acid, but not a double-stranded nucleic acid (See FIG. 1).

U.S. Pat. No. 6,428,959 in the name of the university of California discloses methods of determining the presence of double stranded nucleic acids in a sample (See FIG. 2). In this case, a nucleic acid present in a fluid sample is translocated through a nanopore and the current amplitude through the nanopore is monitored. In this case, a double-stranded nucleic acid can be detected on a current blockade profile.

“[Direct Detection of Anantibody-Antigen Binding Using an On-Chip Artificial Pore PNAS], 100, 820-824 (2003)” presented by Saleh et al. discloses a resistive pulse method of particle sizing with a pore to detect the binding of unlabeled antibodies to the surface of latex colloids (See FIGS. 3A and 3B). In this case, pulses corresponding to the resistance of the pore are measured based on an increase in the size of the bead (bead size=500 nm, pore size: 1 micron), and pressure is used as the driving force.

However, since, in all of the conventional techniques, signals are detected based on the size ratio of a pore to a subject to be measured, materials cannot be precisely separated. In addition, the conventional techniques exhibit low reproducibility and cannot be used to solve the difficulty in manufacturing a pore with a size (<˜10 nm) small enough to separate a single strand and a double strand. As a result, in order to obtain signal separation, high resolution of signal is required. Further, the method disclosed by Sohn et al. cannot be used when a protein small in size or DNA is used.

In order to solve these problems in conventional techniques, the inventors of the present invention have confirmed that biomolecules can be effectively separated and detected by determining the passage of biomolecules through a pore according to magnitude of a charge of a target biomolecule bound to a particle, without being affected by the size of the pore and completed the present invention.

### SUMMARY OF THE INVENTION

The present invention provides a method of effectively separating biomolecules using a nanopore.

The present invention also provides a method of detecting biomolecules separated using the separation method by producing a signal with a high signal to noise ratio.

According to an aspect of the present invention, there is provided a method of separating particles, the method comprising: forming a first chamber and a second chamber separated by an interface with a pore, wherein the first and second chambers have electrodes with different polarities; placing particles to which a target biomolecule is bound from particles to which the target biomolecule is not bound in the first chamber; applying a voltage which has the same charge as that of the target biomolecule to the electrode of the first chamber, and a voltage which has an opposite polarity to that of the target biomolecule to the electrode of the second chamber; and translocating only the particles to which the target biomolecule is bound from the first chamber to the second chamber through the pore.

Each of the first and second chambers may include a vessel or well used to contain a sample and a reaction solution, the interface may include a membrane and a wall used to separate the first chamber and the second chamber, the pore may include a channel connecting the first chamber to the second chamber, and the particle may include a bead with a diameter of mirco or nano meters.

According to the present invention, a voltage with the same charge as that of the target biomolecule is applied to the electrode of the first chamber and a voltage with a polarity opposite to that of the target biomolecule is applied to the electrode of the second chamber so that only the particles to which the target biomolecule is bound is translocated into the second chamber through the pore by an electrical repulsive force from the first chamber and an electrical attractive force in the second chamber.

The particles may be neutral or have a charge opposite to the charge of the electrode of the first chamber because the target biomolecule must remain in the first chamber before binding.

The particle may be any material that can be bound with a biomolecule, and preferably a material selected from glass, metal, a polymer, a protein, a virus, and a dendrimer. The surface of the particle may be made neutral or given an electrical charge opposite to that of the electrode of the first chamber by transforming the particle or controlling the pH of a reaction solution, and preferably is bound with a probe molecule that can be hybridized with the target biomolecule. The probe can be an oligonucleotide when the target molecule is a nucleic acid, and an antibody when the target molecule is a protein.

The target biomolecule may be any biomolecule that can have an electric charge, such as DNA or RNA with a negative charge, or a protein or a peptide with a positive or negative charge. The protein or peptide may have a unique charge, or may have a desired charge by controlling pH of the reaction solution.

In the method, the particle that is bound with the target biomolecule and the particle that is not bound with the target biomolecule can be placed in the first chamber, and preferably, the particles and the target biomolecule are placed in the first chamber, and the same particles are bound or hybridized with the target biomolecule, such that the particle that is bound with the target biomolecule and the particle that is not bound with the target biomolecule exist in the first chamber.

According to another aspect of the present invention, there is provided a method of detecting the target biomolecule by measuring blockades of an ionic current generated through the pore by a current ammeter connected to electrodes when the particle bound with the target biomolecule passes through the pore.

The blockades of the ionic current occur when a portion of the pore is blocked by the particle passing through the pore. The more biomolecules are bound to the particle, more particles may block the pore and the current.

The first and second chambers may be filled with an ionic solution which can generate the ionic current. The ionic solution may be a KCl solution, a NaCl solution, a MgCl₂ solution, or the like, and preferably KCl since K ions and Cl ions have almost same mobility.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a method of detecting a double-stranded nucleic acid disclosed in U.S. Pat. No. 6,362,002 in the name of the university of Harvard;

FIG. 2 illustrates a method of detecting a double-stranded nucleic acid disclosed in U.S. Pat. No. 6,428,959 in the name of the university of California;

FIGS. 3A and 3B illustrate a method of detecting antibody-antigen binding using an artificial pore described in PNAS, 100, 820-824 (2003);

FIGS. 4A and 4B illustrate the movement of a particle before and after hybridization according to an embodiment of the present invention;

FIGS. 5A and 5B illustrate various particles that can be detected according to an embodiment of the present invention;

FIG. 6A is a transmitting electron microscopy (TEM) image of a pore formed in Example 1;

FIG. 6B is a scanning electron microscopy (SEM) image of beads formed in Example 1; and

FIG. 7 is sequential images illustrating beads moving across electrode plates as a result of the movement induced by controlling the electric charge of the beads by binding the beads with DNA according to an embodiment of the present invention.

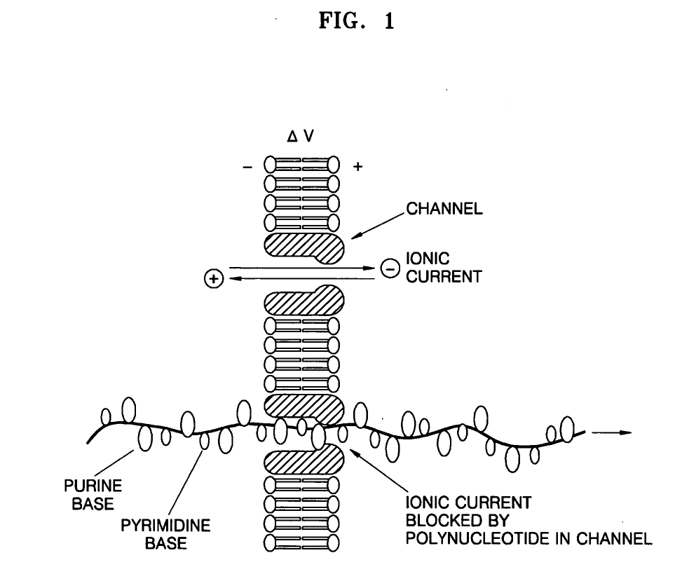

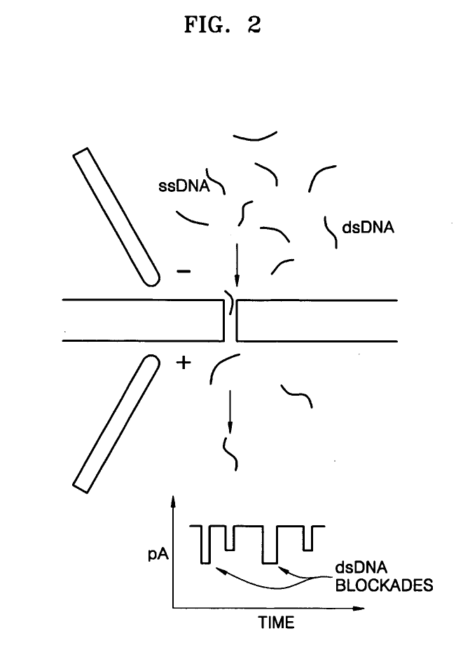

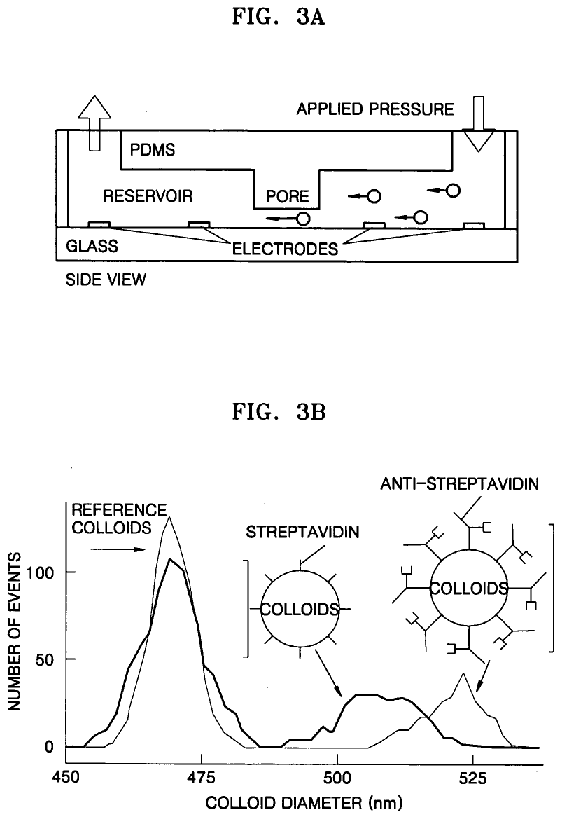

### DETAILED DESCRIPTION OF THE INVENTION

Hereinafter, the present invention will be described in detail with reference to the accompanying drawings.

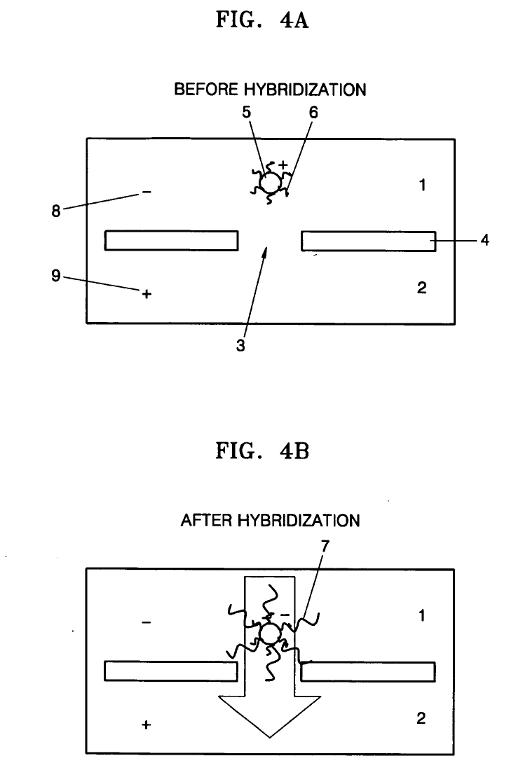

FIGS. 4A and 4B illustrate the movement of a particle before and after hybridization according to an embodiment of the present invention. An apparatus according to an embodiment of the present invention includes a first chamber 1 with an electrode 8, a second chamber 2 with an electrode 9 with an opposite charge to the electrode 8, an interface 4 having a pore 3 between the first chamber 1 and the second chamber 2, and a particle that can be bound with a target biomolecule 7 or that is bound with a probe 6 to which the target biomolecule 7 can be bound. Referring to FIG. 4A, before hybridization, the particle 6 has an electric charge opposite to the first chamber 1 is a neutral so that the particle 6 remains in the first chamber 1. Referring to FIG. 4B, after the hybridization, the particle 5 bound with the target biomolecule 5 and the first chamber 1 have identical electric charges so that the particle 5 is repelled from the first chamber 1 and moves to the second chamber 2 through the pore 3. This movement is physical separation caused by the net charge of the particle 5, which is different from signal separation. In detail, when the biomolecule 5 is DNA, the bead including a capture probe has a neutral or small positive electric charge, the ionic solution includes 1 M KCl for generation of an ionic current, the size of the pore 3 is about 100 nm, and the minimum size of a bead is 40 nm. However, DNA cannot be detected using conventional methods when the size of the pore is about 100 nm.

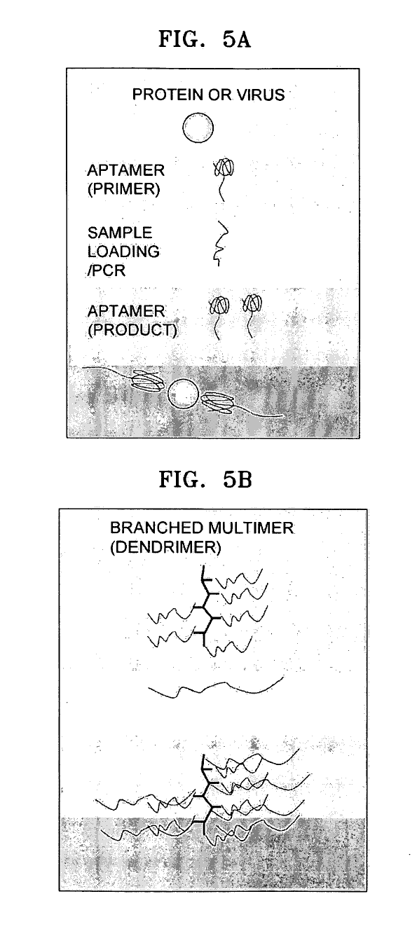

FIGS. 5A and 5B illustrate various particles that can be detected according to an embodiment of the present invention. Referring to FIG. 5A, a protein or virus, instead of beads, can be used as the particle. In this case, a sample can be PCR amplified using a primer with an aptamer that can be bonded to the surface of the particle and the PCR product with a plurality of aptamers can be bound with the particle. Referring to FIG. 5B, instead of the bead, dendrimer, which is a branched multimer, can be used as the particle. In this case, biomolecules can be bound to each branch of the dendrimer. Examples of a positive dendrimer that can be used for gene transmission include a polyamidoamine (PAMAM) dendrimer, a polypropylene imine (PPI) dendrimer, a poly L-lysine (PLL) dendrimer, and the like.

The present invention will now be described more fully with reference to the following examples. The examples are provided for illustrative purpose only and are not intended to limit the scope of the invention.

### EXAMPLE 1

### Manufacture of Separating Apparatus According to an Embodiment of the Present Invention

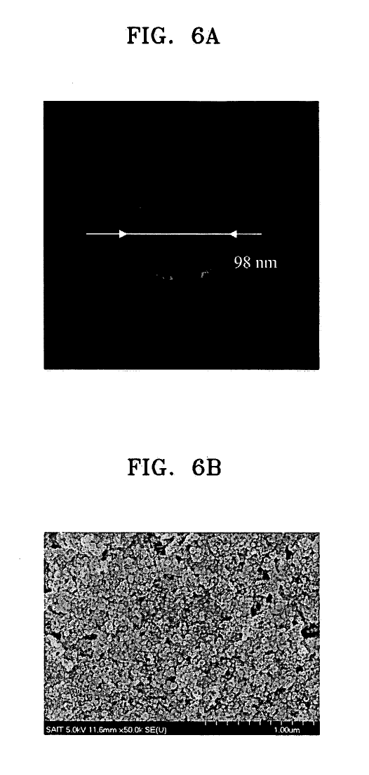

Referring to FIGS. 4A and 4B, in order to separate and detect DNA, a silicated bead (SS-SOL30FH2 obtained from Shinheung silicate Co., Ltd.) with a diameter of 45 nm was reacted with an r-Aminopropyltriethoxysilane (APTES) solution in an ethanol aqueous solution at room temperature for one hour, thus producing a nano bead coated with amine (in order to obtain a positive surface charge). Then, a silicon nitride membrane with a diameter of 100 nm was manufactured using a standard lithography method and pores with a diameter of about 100 nm were formed in the silicon nitride membrane using a focused ion beam. FIG. 6A is a transmission electron microscopy (TEM) image of a pore and FIG. 6B is a scanning electron microscopy (SEM) image of beads. When the size of the bead increases due to binding with DNA, the a multitude of a blockade signal increases, and only the bead bound with DNA passes through the pore when a voltage is applied as illustrated in FIG. 4 to produce the blockade signal. The Blockade signal is given by

$$\vert \frac{δ I}{I} \vert = \frac{D}{L} [ \frac{arcsin ( d / D )}{\sqrt{1 - {( d / D )}^{2}}} - \frac{d}{D} ] ,$$

where L is the diameter of a pore, d is the diameter of a particle, and D is the diameter of a pore.

### EXAMPLE 2

### Control of Electric Charge of Bead by Binding with DNA

In order to confirm that the movement of the bead is caused by controlling the electric charge of the bead by binding with DNA, a silicate bead with a diameter of 45 nm was coated with amine using r-Aminopropyltriethoxysilane (APTES) and DNA was fixed onto the coated silicate bead. Beads fixed with various concentrations of DNA were placed in a cell and experiments were performed.

1. Manufacture of Bead

An amine modified silicate bead with a diameter of 45 nm was coated with APTES in an ethanol solvent at STP for one hour.

2. DNA Fixation

Material: 5′-CTTGGTCTGTATGACATCTAAAT-3′

Concentration: 0, 10, 100, 250 nM

Conditions: DNA+bead (mixing): 70° C., 1.5 hours blocking after fixation: succinic anhydride (10 minutes)

3. Measurement

Applied voltage: 0.2 V/2 mm

Applied time: 1 min

Electrode type: Au+Au with Cr layer as adhesion layer

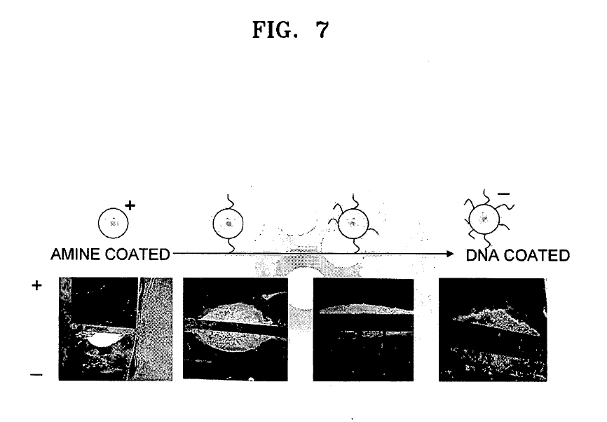

FIG. 7 is sequential images illustrating beads moving accross electrode plates as a result of controlling the electric charge of the beads by binding the beads with DNA. Referring to FIG. 7, it was confirmed that the electric charge of the bead can be controlled by the concentration of DNA bound to the surface of the bead, thus inducing the movement of the bead.

As described above, conventionally, the size of a pore is used to separate biomolecules. However, effective separation is difficult to achieve because the manufacture of a pore with a diameter of less than 10 nm, small enough to separate biomolecules is not easy. Therefore, signal separation and data analysis are required. However, in the present method, physical movement induced by the charge of biomolecules is used to effectively separate the biomolecules, thus obtaining a high signal to noise ratio. As a result, additional data analysis is not required.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of separating particles, the method comprising:
forming a first chamber and a second chamber separated by an interface with a pore, wherein the first and second chambers have electrodes with different polarities;
placing particles to which a target biomolecule is bound from particles to which the target biomolecule is not bound in the first chamber;
applying a voltage which has the same charge as that of the target biomolecule to the electrode of the first chamber, and a voltage which has an opposite polarity to that of the target biomolecule to the electrode of the second chamber; and
translocating only the particles to which the target biomolecule is bound from the first chamber to the second chamber through the pore.

2. The method of claim 1, wherein the particles are neutral, or have a charge opposite to the charge of the electrode of the first chamber.

3. The method of claim 1, wherein the particles are selected from the group consisting of glass, metal, a polymer, a protein, a virus, and a dendrimer.

4. The method of claim 1, wherein the particle is bound with a probe molecule such that the particle is hybridized with the target biomolecule.

5. The method of claim 1, wherein the target biomolecule is DNA or RNA with a negative charge, or a protein or a peptide with a positive or negative charge.

6. The method of claim 1, wherein the placing of the particles in the first chamber is carried out by binding or hybridizing the target biomolecule to the particles in the first chamber.

7. A method of detecting a target biomolecule comprising: passing only the particle to which the target biomolecule is bound through the pore using the separation method of claim 1: and measuring blockades of an ionic current generated through the pore by a current ammeter connected to the electrodes.

8. The method of claim 7, wherein the first and second chambers are filled with an ionic solution which can generate the ionic current.


SEQUENCE LISTING (1 sequence)

SEQ ID NO: 1 - DNA, 23 bp, artificial sequence
  synthetic construct
cttggtctgtatgacatctaaat